(19) United States
(12) Patent Application Publication
(10) Pub. No.: US 2010/0240129 A1
(43) Pub. Date: Sep. 23, 2010

(54) Transfer of Molecules

(76) Inventor: Michael Patane, Seaforth (AU)

Correspondence Address: Ballard Spahr LLP, SUITE 1000, 999 PEACHTREE STREET, ATLANTA, GA, 30309-3915, US

(21) Appl. No.: 12/791,076

(22) Filed: Jun. 1, 2010

Related U.S. Application Data
(63) Continuation of application No. 11/667,421, filed on May 9, 2007, now abandoned.

(30) Foreign Application Priority Data
Dec. 16, 2004 (AU) 2004907178

(51) Int. Cl. C12N 5/02

(52) U.S. Cl. 435/375

(57) ABSTRACT

The invention relates to liposomes, particularly, but not exclusively, to liposomes for introducing a molecule into a cell, and to processes for making and using liposomes.

### FIELD OF THE INVENTION

The invention relates to liposomes, particularly, but not exclusively, to liposomes for introducing a molecule into a cell, and to processes for making and using liposomes.

### BACKGROUND OF THE INVENTION

A liposome is a structure comprising one or more concentric spheres of a lipid bilayer that enclose an aqueous compartment. Liposomes have a diameter in the range of 50 nm to 200 μm and are typically prepared by the process of dispersing lipids in an organic solvent, re-dispersion or hydration of the lipids in an aqueous medium to form a lipid vesicle, and size reduction and purification from the aqueous medium.

As the lipid bilayer of a liposome is inherently impermeable to ions and most polar molecules, liposomes can be used to contain molecules, for example, by encapsulating them. Examples of such molecules include proteins, nucleic acids and compounds such as pharmaceuticals. Accordingly, liposomes find particular application in the delivery of molecules to a biological system where they are useful for protecting molecules from degradation and for targeting molecules to particular cells or tissues.

The fusion, or in other words, lipid exchange of the liposome lipid bilayer with a cellular lipid bilayer after endocytosis, particularly an endosome lipid bilayer, is an important step in the transfer of a molecule contained by a liposome into a cell. Accordingly, it is believed that liposome adaptations that improve the fusion of the liposome to a cellular lipid bilayer are important for improving the efficiency of transfer of a molecule contained by a liposome to a cell.

pH-sensitive liposomes have been created that are adapted to destabilise at the pH of an endosome and so favour the fusion of the liposome lipid bilayer with the endosome lipid bilayer. These and other adaptations have typically provided limited improvement in the fusion of a liposome lipid bilayer to a cellular lipid bilayer, and accordingly to improving the efficiency of transfer of a molecule contained by a liposome to a cell, as evidenced by the fact that to date it has not been possible to use liposomes to deliver molecules to many cells. For example, liposomes have had limited application in drug delivery to date.

In summary, there is a need for liposomes with an improved capacity for fusion with a target lipid bilayer.

### SUMMARY OF THE INVENTION

In one aspect there is provided a process for producing a liposome capable of fusing with a target lipid bilayer. The process comprises identifying species of lipids of the target lipid bilayer, measuring the amounts of each identified species in the target lipid bilayer as a function of the total amount of molecular species of the target lipid bilayer, selecting lipids of the identified species and preparing a liposome comprising the selected lipids, in the measured amounts, to produce the liposome.

In another aspect, there is provided a process for producing a liposome capable of fusing with a target lipid bilayer of an animal or plant cell. The process comprises identifying the species of phosphatidyl choline and phosphatidyl ethanolamine molecules of the target lipid bilayer, measuring the amounts of each identified species in the target lipid bilayer as a function of the total amount of molecular species of the target lipid bilayer, selecting phosphatidyl choline and phosphatidyl ethanolamine molecules of the identified species and preparing a liposome comprising a sterol and the selected phosphatidyl choline and phosphatidyl ethanolamine molecules, in the measured amounts, to produce the liposome.

In another aspect, there is provided a process for selecting lipids for forming a liposome capable of fusing with a target lipid bilayer. The process comprises identifying the species of lipids of the target lipid bilayer, measuring the amounts of each identified species in the target lipid bilayer as a function of the total amount of molecular species of the target lipid bilayer and selecting the identified species of lipids, in the measured amounts, to select lipids for forming the liposome.

In another aspect, there is provided a process for determining whether a composition of lipids is capable of forming a liposome for fusing with a target lipid bilayer. The process comprises identifying the species of lipids of the target lipid bilayer, measuring the amounts of each identified species in the target lipid bilayer as a function of the total amount of molecular species of the target lipid bilayer and determining whether the composition comprises the identified species of lipids, in the measured amounts. In other words, the amounts of the identified species in the target lipid bilayer is compared with amounts of the identified species in the composition.

In another aspect, there is provided a process for determining whether a liposome is capable of fusing with a target lipid bilayer. The process comprises identifying the species of lipids of the target lipid bilayer, measuring the amounts of each identified species in the target lipid bilayer as a function of the total amount of molecular species of the target lipid bilayer and determining whether the liposome comprises the identified species of lipids, in the measured amounts. In other words, the amounts of the identified species in the target lipid bilayer is compared with the amounts of the identified species in the liposome.

In another aspect, there is provided a process for selecting a liposome for fusing with a target lipid bilayer. The process comprises identifying the species of lipids of the target lipid bilayer, measuring the amounts of each identified species in the target lipid bilayer as a function of the total amount of molecular species of the target lipid bilayer and determining whether the liposome comprises the identified species of lipids, in the measured amounts, to select the liposome.

In another aspect there is provided a process for producing a liposome for fusing with a target lipid layer comprising:

a) analysing a target lipid layer to determine the identity of species of lipids comprised in the target lipid layer;

b) measuring the amount of each identified species of lipid in the target lipid layer to determine the relative amount of each identified species in the target lipid layer; and

c) combining lipids of each identified species to form a liposome in which each identified species has a relative amount that is substantially the same as in the target lipid layer.

In another aspect, there is provided a liposome for fusing with a target lipid bilayer. The liposome comprises a lipid bilayer having a relative amount of a species of lipid that is the same as the relative amount of the species of lipid in the target lipid bilayer. The liposome may further comprise binding means for binding the liposome to the target lipid bilayer. Typically the liposome is adapted for destabilisation of the liposome lipid bilayer at a pH about 5 to 7.0.

In another aspect, the invention provides a liposome for fusing with a target animal or plant cell. The liposome comprises a lipid bilayer having relative amounts of species of phosphatidyl choline, phoshpatidyl ethanolamine and a sterol that are the same as the relative amounts of the species in the target animal or plant cell lipid bilayer. The liposome may further comprise binding means for binding the liposome to the animal or plant cell. Typically the liposome is adapted for destabilisation of the liposome lipid bilayer at a pH of an early endosome of the target animal or plant cell.

The invention also provides a composition comprising a liposome according to the invention.

### DETAILED DESCRIPTION OF THE EMBODIMENTS

As described herein, the inventor has found that the fusion of the lipid bilayer of a liposome and a target lipid bilayer, for example, a lipid bilayer of a target cell such as a plasmalemma lipid bilayer or an endosomal lipid bilayer, is favoured and provides for improved transfer of a molecule contained by the liposome, where the liposome lipid bilayer has the same species of lipids as the target lipid bilayer and in the same relative amounts as the target lipid bilayer.

Importantly, the inventor has found that a liposome having a lipid bilayer that has the same species of lipids as the target lipid bilayer, and in the same relative amounts as the target lipid bilayer, is capable of fusing with a membrane of a target cell to permit transfer of a molecule contained by the liposome to an early endosome. This is an important finding because the early endosome of the endocytosis pathway is recognised as being a key site for transfer of molecules into a cell cytoplasm, especially for permitting the transferred molecule to exert the desired effect. Prior to the invention, it had been difficult to cause a liposome to transfer molecules contained by the liposome into an early endosome. Indeed, liposomes available prior to the invention had often been observed to be contained in cell organelles such as late endosomes and lysosomes, from which transfer of molecules in a functional form to the cell cytoplasm is not possible.

Thus the invention provides a process for producing a liposome capable of fusing with a target lipid bilayer. The process comprises the following steps:

(a) identifying species of lipids of the target lipid bilayer;

(b) measuring the amounts of each identified species in the target lipid bilayer as a function of the total amount of molecular species of the target lipid bilayer;

(c) selecting lipids of the identified species; and

(d) preparing a liposome comprising the selected lipids in the measured amounts, to produce the liposome.

The relative amount of a species in the target lipid bilayer or the liposome lipid bilayer is the amount of the species as a function of the total amount of molecular species of the bilayer. Accordingly, the percentage amount of a species of the target lipid bilayer, or liposome lipid bilayer, is a measure of the relative amount of that species, or in other words, the amount of that species as a function of the total amount of molecular species of the bilayer.

It will be understood that the lipid bilayer of the liposome does not need to be identical to the lipid bilayer of the target lipid bilayer to favour fusion of the target lipid bilayer and the liposome bilayer. In other words, fusion of the lipid bilayers of the liposome and the target is favoured when the liposome lipid bilayer comprises the species of lipids of the target lipid bilayer that have the greater abundance in the target lipid bilayer. Thus, in one embodiment, step (c) of the process comprises selecting lipids of an identified species that have greater abundance in the target lipid bilayer. In another embodiment, step (c) comprises selecting lipids of about 5 identified species that together constitute about 80% of the lipids of the target lipid bilayer. In another embodiment, step (c) comprises selecting lipids of 2 identified species that together constitute about 80% of the lipids of the target lipid bilayer.

It is important that the relative amounts of the selected species in the liposome lipid bilayer should be about the same as the relative amounts in the target lipid bilayer, however the relative amounts do not need to be identical. For example, as described herein, where a lipid is identified in a target lipid bilayer in a relative amount of 50 molar %, the liposome lipid bilayer may comprise between about 40-50% of that lipid.

It is believed that improvements in the fusion of the liposome and the target lipid bilayers can be optimised by preparing the liposome so that the selected lipids are arranged on a layer of the liposome lipid bilayer that corresponds with the layer of the target lipid bilayer. This layer of the liposome lipid bilayer is referred to herein as a corresponding layer. More particularly, as described herein, where phoshpatidyl ethanolamine is identified as being comprised in the outer layer of a target cell bilayer (i.e. the layer exposed to the extracellular environment), the liposome is prepared so that the phosphatidyl ethanolamine is located on the outer layer of the liposome (i.e. the layer for contact with the target cell bilayer). In this circumstance, the outer layer of the liposome is the corresponding layer of the liposome lipid bilayer. As a further example, where a species is identified to be arranged on an inner layer of the target bilayer (i.e. the layer that is in contact with a cytoplasm), the liposome is to be prepared so that the identified species is arranged on the inner layer of the liposome lipid bilayer (i.e. the layer that is in contact with the aqueous compartment of the liposome). In this circumstance, the inner layer of the liposome lipid bilayer is the corresponding layer.

The inventor has found that fusion of the target lipid bilayer and liposome lipid bilayer of the invention is further optimised to permit improved transfer of a molecule contained by a liposome to a target where the liposome comprises a binding means for binding the liposome to the target lipid bilayer. For example, as described herein, the inventor has found that the use of 1,2-dioleoyl-sn-glycero-3-trimethylammonium-propane (DOTAP) in liposome lipid bilayers made according to the invention improves transfer of a molecule contained by a liposome to a target cell. DOTAP is a cationic compound that provides a positive charge to a liposome. Thus, in one embodiment of the process, in step (d) a liposome is prepared further comprising a binding means for binding the liposome to the target lipid bilayer. Typically the binding means is a compound for providing the liposome with a charge for attracting the liposome to the target lipid bilayer, in particular a positive charge. Alternatively, the binding means may be a compound capable of aggregating a liposome to a target lipid bilayer, such as a compound comprising glycerol. Still further, the binding means may be a molecule capable of selectively binding the liposome to the target lipid bilayer. Examples of such molecules include antibodies and ligands capable of interacting selectively with a receptor, for example, such as is observed in the biotin-streptavidin interaction.

The process of the invention further comprises the step of adapting the liposomes produced by the process so as to be pH sensitive, or in other words, to be sensitive to a particular pH range such that the liposome lipid bilayer is caused to destabilise, or in other words, to disrupt at that pH range. Thus typically in step (d), a liposome is adapted for destabilisation of the liposome lipid bilayer at the pH of the target lipid bilayer. In one embodiment, the liposome is adapted for destabilisation of the liposome lipid bilayer at a pH of between about 5.0 and 7.0.

Typically, the above described process is for producing a liposome capable of fusing with a target lipid bilayer of a cell, or a lipid bilayer derived from, or in other words, obtained or extracted from a cell. However, it follows that the process is suitable for producing a liposome capable of fusing with an artificial, or in other words, a synthetically derived lipid bilayer. Thus in one embodiment, the process is for producing a liposome for fusing with a synthetically derived target lipid bilayer. It will be understood that the synthetically derived target lipid bilayer may be a liposome.

The invention also provides a process for producing a liposome capable of fusing with a target lipid bilayer of an animal or plant cell. The process comprises:

(a) identifying the species of phosphatidyl choline and phosphatidyl ethanolamine molecules of the target lipid bilayer;

(b) measuring the amounts of each identified species in the target lipid bilayer as a function of the total amount of molecular species of the target lipid bilayer;

(c) selecting phosphatidyl choline and phosphatidyl ethanolamine molecules of the identified species; and

(d) preparing a liposome comprising sterol and the selected phosphatidyl choline and phosphatidyl ethanolamine molecules, in the measured amounts, to produce the liposome.

Although phosphatidyl choline and phosphatidyl enthanolamine are typically the most abundant phospholipids in an animal or plant cell lipid bilayer, the inventor has observed that the species of the phosphatidyl choline and phosphatidyl ethanolamine, with respect to the nature of the acyl chain of these phospholipids, is an important consideration in the process for preparing the liposomes of the invention. Typically, the selected phoshpatidyl choline and phosphatidyl ethanolamine molecules have an acyl chain comprising at least 14 carbon atoms, typically 16 to 18 carbon atoms, although longer chains are within the scope of the invention. Further, the selected phosphatidyl choline and phosphatidyl enthanolamine molecules have an acyl chain that is typically unsaturated, or in other words, the acyl chain comprises at least one double bond.

An example of a useful sterol is cholesterol and/or ergosterol.

In accordance with the first described aspect of the invention, the lipid bilayer of the liposome does not need to be identical to the target lipid bilayer of the animal or plant cell, with respect to the type and relative amount of lipid comprised in the liposome lipid bilayer. However, it will be understood that with greater similarity between the liposome and target lipid bilayers, the improvement in fusion of liposome and target cell lipid bilayers may be optimised.

One approach for producing a liposome having lipid bilayers with similarity to the lipid bilayers of the target cell is to prepare the liposome of step (d) of the invention so that the identified species are arranged on a layer of the liposome lipid bilayer that corresponds with the layer of the target cells, as noted above in accordance with the first described aspect of the invention.

Alternatively or additionally, another approach is to prepare the liposome of step (d) of the invention so that the liposome comprises components of the target cell lipid bilayer other than phosphatidyl choline, phoshpatidyl ethanolamine and a sterol. In this regard it is noted that phospholipids comprising an alcohol other than choline or enthanolamine may be included in the lipid bilayer of an animal or plant cell, albeit in less abundance than phosphatidyl choline and phosphatidyl enthanolamine. Thus in one embodiment of the process, step (d) further comprises preparing a liposome comprising a phospholipid comprising an alcohol selected from the group consisting of serine, inositol and glycerol.

Further, it is noted that phospholipids comprising sphingosine instead of glycerol are also observed in the lipid bilayer of an animal or plant cell. Thus in another embodiment of the process, step (d) further comprises preparing a liposome comprising a phospholipid comprising sphingosine.

Glycolipids and glycoproteins may also be comprised in the lipid bilayer of an animal and plant cell. Accordingly, in another embodiment, step (d) further comprises preparing a liposome comprising a phospholipid comprising a sugar. In another embodiment, step (d) further comprises preparing a liposome comprising a protein capable of being arranged in a lipid bilayer of the liposome.

Examples of animal cells to which the process and liposomes of the invention may be targeted include cells that are located on surfaces, and especially epithelial cells. Examples of include epithelial cells that function in covering and lining surfaces (such as skin epithelia), absorption (such as the intestinal epithelia), secretion (such as glandular epithelia), sensation (such as neuroepithelia) and contractility (such as myoepithelia). These cells may be present in a single layer (simple) or in 2 or more layers (stratified) or they may be psuedostratified. They may be columnar epithelia, as found in intestine, cuboidal epithelia, as found in kidney and squamous epithelia, as found in cornea. Other complex types of epithelia include stratified squamous keratinized and non keratinized epithelia, transitional epithelia and pseudostratified columnar ciliated epithelia.

Of particular relevance are the stratified squamous epithelia of the oral cavity and in particular the oral epithelium located on filiform, fungiform, foliate and circumvallate papillae.

The invention also provides a liposome produced by the above described processes of the invention. These liposomes are described further below.

It will be understood that the invention has particular utility for the selection of lipids for forming liposomes. Accordingly, it will be understood that in certain applications, a process of the invention does not include the step (d) of preparing a liposome. Thus, the invention provides a process for selecting lipids for forming a liposome capable of fusing with a target lipid bilayer. The process comprises:

(a) identifying the species of lipids of the target lipid bilayer;

(b) measuring the amounts of each identified species in the target lipid bilayer as a function of the total amount of molecular species of the target lipid bilayer; and

(c) selecting the identified species of lipids, in the measured amounts, to select lipids for forming the liposome.

Further, it will be understood that the processes of the invention have particular utility where a composition of lipids or liposomes is available and a determination is required on whether such components would be capable of fusing with a particular target lipid bilayer. Thus, the invention also provides a process for determining whether a composition of lipids is capable of forming a liposome for fusing with a target lipid bilayer. The process comprises:

(a) identifying the species of lipids of the target lipid bilayer;

(b) measuring the amounts of each identified species in the target lipid bilayer as a function of the total amount of molecular species of the target lipid bilayer; and

(c) determining whether the composition comprises the identified species of lipids, in the measured amounts, to determine whether the composition is capable of forming a liposome for fusion with the target lipid bilayer.

Also provided is a process for determining whether a liposome is capable of fusing with a target lipid bilayer. The process comprises:

(a) identifying the species of lipids of the target lipid bilayer;

(b) measuring the amounts of each identified species in the target lipid bilayer as a function of the total amount of molecular species of the target lipid bilayer; and

(c) determining whether the liposome comprises the identified species of lipids, in the measured amounts, to determine whether the liposome is capable of forming a liposome for fusion with the target lipid bilayer.

The processes of the invention also have particular utility for selecting a liposome for a particular capacity for fusing with a target lipid bilayer. Thus, the invention also provides a process for selecting a liposome for fusing with a target lipid bilayer. The process comprises:

(a) identifying the species of lipids of the target lipid bilayer;

(b) measuring the amounts of each identified species in the target lipid bilayer as a function of the total amount of molecular species of the target lipid bilayer; and

(c) determining whether the liposome comprises the identified species of lipids, in the measured amounts, to select the liposome.

In another aspect there is provided a process for producing a liposome for fusing with a target lipid layer comprising:

a) analysing a target lipid layer to determine the identity of species of lipids comprised in the target lipid layer;

b) measuring the amount of each identified species of lipid in the target lipid layer to determine the relative amount of each identified species in the target lipid layer; and

c) combining lipids of each identified species to form a liposome in which each identified species has a relative amount that is substantially the same as in the target lipid layer.

In one embodiment, in step (a), the target lipid layer is analysed to determine the identity of all species of lipids comprised in the target lipid layer.

In another embodiment, in step (c), the lipids of all identified species are combined to form a liposome. In other embodiments only those species that are most abundant in the target lipid layer are combined, for example the lipids of more than one identified species that together constitute about 80% of the lipids of the target lipid layer are combined to form a liposome.

In a further embodiment, at least one identified species of lipid is phosphatidyl choline or phosphatidyl ethanolamine.

In another embodiment, at least one sterol is combined with lipids of each identified species in step c) to form a liposome.

With reference to the above described processes, it will be understood that the lipid bilayer of the liposome does not need to be identical to the lipid bilayer of the target lipid bilayer to favour fusion of the target lipid bilayer and the liposome bilayer. In other words, fusion of the lipid bilayers of the liposome and the target is favoured when the liposome lipid bilayer comprises the species of phospholipids and sterols of the target lipid bilayer that have the greater abundance in the target lipid bilayer.

Further, it is important that the relative amounts of the selected species in the liposome lipid bilayer should be about the same as the relative amounts in the target lipid bilayer, however the relative amounts do not need to be identical. For example, where a lipid is identified in a target lipid bilayer in a relative amount of 50 molar %, the liposome lipid bilayer may comprise between about 40-50 molar % of that lipid.

With reference to the above described processes, in accordance with the invention, the species of lipids of the target lipid bilayer, including phospholipids and sterols, can be identified and determined by standard techniques known to one skilled in the art including nuclear magnetic resonance, gas liquid chromatography, gas chromatography mass spectrometry, high pressure liquid chromatography and thin layer chromatography.

Further, liposomes may be prepared by standard techniques known to one skilled in the art. Examples of suitable techniques are the dehydration/rehydration technique, the reverse phase evaporation technique, the ethanol injection technique, the detergent dialysis technique, the sonication technique, the microfluidizer technique, the extrusion technique and the French press technique. It will be understood that, where applicable, the selection of a particular technique for preparation of the liposome will be dependent on the purpose for which the liposome is to be put, i.e. the purpose for which the liposome is to be fused to the target lipid bilayer.

The invention also provides a liposome for fusion with a target lipid bilayer. The liposome comprises a lipid bilayer having:

(a) a relative amount of a species of lipid that is the same as the relative amount of the species of lipid in the target lipid bilayer; and

(b) binding means for binding the liposome to the target lipid bilayer.

Typically the liposome is adapted for destabilisation of the liposome lipid bilayer at a pH below about 7.0.

With reference to the liposomes of the invention, the relative amount of a species in the liposome lipid bilayer, or in the target lipid bilayer or a bilayer of a target cell, is the amount of the species as a function of the total amount of molecular species of the bilayer. Accordingly, the percentage amount of a species of a lipid bilayer is a measure of the relative amount of that species, or in other words, the amount of that species as a function of the total amount of molecular species of the bilayer.

With reference to the liposomes of the invention, it will be understood that the lipid bilayer does not need to be identical to the lipid bilayer of the target lipid bilayer or a bilayer of a target cell to favour fusion of lipid bilayers. In other words, fusion of the lipid bilayers of the liposome and the target is favoured when the liposome lipid bilayer comprises the species of phospholipids and sterols of the target that have the greater abundance in the target lipid bilayer or bilayer of a target cell.

Typically, the lipid bilayer of the liposome of the invention comprises more than one species of lipid. For example, depending on the species of lipid comprised in the target lipid bilayer, the lipid bilayer of the liposome may comprise species of lipid selected from the group consisting of phospholipids, sphingomyelin and sterols. Where the lipid bilayer of the liposome comprises more than one species of lipid, each of the more than one species of lipid has a relative amount in the liposome bilayer that is the same as the relative amount of each of the more than one species of the target lipid bilayer.

With reference to the liposomes of the invention, it will be understood that it is important that the relative amounts of the selected species in the liposome lipid bilayer should be about the same as the relative amounts in the target lipid bilayer, however the relative amounts do not need to be identical.

Typically, the liposome of the invention has a pH sensitivity of about pH7.0, which means that the liposome is unstable below pH7.0 such that the lipid bilayer of the liposome is disrupted below pH7.0 In one embodiment, the liposome has a pH sensitivity of about 5.5 to 6.0, and accordingly, the lipid bilayer is disrupted in this pH range.

The liposome may have a diameter ranging between about 50 nm and 200 μm. Accordingly, the liposome may be a small, sonicated unilamellar vesicle (SUV), a large unilamellar vesicle (LUV), or a liposome prepared by reverse phase evaporation (a REV), by french press (a FPV) or by ether injection (an EIV). Methods of preparing liposomes of such sizes, including methods of fractionating and purifying liposomes of the desired size, are known to one skilled in the art. Typically, the liposome has a diameter ranging between about 100 and 600 nm. In one embodiment, the liposome has a diameter of between about 200 and 400 nm. In one embodiment, the liposome is a LUV.

Typically, the liposome of the invention is a unilamellar with respect to the liposome lipid bilayer. However, it will be understood that the liposome of the invention may comprise more than one lipid bilayer, provided that the lipid bilayer for contact with the target lipid bilayer has a relative amount of a species of lipid that is the same as the relative amount of the species of lipid in the target lipid bilayer. Thus in one embodiment, the liposome is a multilamellar vesicle such as a large, vortexed multilamellar vesicle (MLV).

As described herein a compound for providing the liposome with a charge for binding the liposome to a target cell is advantageous for improving the fusion between the target lipid bilayer and the liposome bilayer. For example, DOTAP is particularly useful as a binding means for binding the liposome lipid bilayer to a target cell. Thus, typically the binding means for binding the liposome to the target lipid bilayer is a compound for providing the liposome with a charge for binding the liposome to the target lipid bilayer. The compound typically provides the liposome with a positive charge. In one embodiment, the compound is DOTAP.

While the binding means is typically a compound for providing the liposome with a charge, it will be understood that the binding means may be another molecular species, such as a monoclonal antibody for binding to a protein on the surface of the target lipid bilayer, or a ligand-receptor pair, such as the biotin-streptavidin receptor pair. Alternatively, the binding means may be a compound for aggregating the liposome on the target, such as a compound comprising glycol. Typically, the binding means is a compound for providing the liposome with a charge and the liposome may further comprise a molecular species for binding the liposome to a target lipid bilayer, such as an antibody.

As described further below, the liposomes of the invention are particularly useful for transferring a compound contained by the liposome into a plant or animal cell. Accordingly, the invention provides a liposome for fusion with a target animal or plant cell. The liposome comprises:

(a) a bilayer having relative amounts of species of phosphatidyl choline, phoshpatidyl ethanolamine and a sterol that are the same as the relative amounts of the species in the target animal or plant cell lipid bilayer; and

(b) binding means for binding the liposome to the animal or plant cell.

Typically the phospholipid comprises an acyl chain having at least 14 carbon atoms. Preferably, the chain has at least 16-18 atoms. Typically, the phospholipid comprises an acyl chain that is unsaturated.

Typically, the sterol is cholesterol and/or ergosterol.

As described below, a compound for providing a liposome with a charge, particularly a positive charge, such as DOTAP, is believed to be useful for binding the liposome to an animal or plant cell. Thus, typically the binding means is a compound for providing the liposome with a positive charge, such as DOTAP, however, it will be understood that the liposome may further comprise a binding means such as a monoclonal antibody for binding to a protein on the target cell surface, or a ligand capable of binding to a receptor on the target cell surface.

Typically the liposome is adapted for destabilisation of the liposome lipid bilayer at the pH of an early endosome. An early endosome is a vacuole or vesicle formed at an early stage of the endocytotic pathway. This organelle typically has a pH ranging from 5.5 to 6.0.

Where the liposome is to be used for the purpose of transferring a molecule contained by the liposome into a cell, the liposome has a size sufficient for endocytocysis. For example, the liposome has a diameter in the range of 200 to 400 nm.

The liposomes of the invention have particular utility in transferring, or in other words, delivering, a compound across a target lipid bilayer, for example, a cell membrane. The compound may be encapsulated by the liposome by being contained an aqueous compartment defined by the liposome lipid bilayer. Thus, in one embodiment, the liposome of the invention further comprises a compound to be delivered across the target lipid bilayer when the liposome is fused with the target lipid bilayer. One example of such a compound is a pharmaceutical. Other examples include food additives, nutraceuticals or the like.

It will be understood that the compound may a molecule that spans the one or more lipid layers of the liposome lipid bilayer. For example, the molecule may be a protein. Further, the compound may protrude from the surface of the liposome lipid bilayer for fusion with the target lipid bilayer. Examples of such molecules are carbohydrates, in the form of glycolipids.

### EXPERIMENTAL

### Materials

Soy phosphatidylcholine (PC), dioleoylphosphatidylethanolamine (DOPE), soy phosphatidylethanolamine (PE), 1,2-dioleoyl-sn-glycero-3-trimethylammonium-propane (DOTAP), ergosterol (ERGO), calcein, fluorescein isothiocyanate dextran, (FITC-FD 250S), dextrose, trisma base, ficoll-type 70, fluorinert FC-77, melting point bath oil, I-sucrose-ul-14C, silicone oil AR 200, lyticase and β-glucuronideas—type H2 were all obtained from Sigma Aldrich (Castle Hill, Australia). Sepharose CL-4B was obtained from Amersham Biosciences (Castle Hill, NSW). Sorbitol was purchased from Med-Chem (Kew, Vic). Yeast extract and bacterial peptone were purchased from Oxoid Chemicals (Heidelberg, Vic), Saccharomyces cerevisiae (YNN 281) was kindly provided by Food Science Australia (North Ryde NSW). All remaining chemicals and solvents were of HPLC grade.

Equipment

A Buchi rotary evaporator and Heto FD-3 freeze drier were used to initially dry and then remove all traces of organic solvents from the prepared phospholipid films with rehydration occurring in a Braun Certomat WT temperature controlled shaking water bath. Formation of multilamellar vesicles (MLV) was initially undertaken in a Braun 1200 bath sonicator with the addition of 2 mm glass beads to aid in the removal of the dried lipid from the walls of the flask. Size reduction of the liposomes from MLVs to large unilamellar vesicles (LUV) was undertaken in an Avestin Lipo-fast basic with stabiliser extruder incorporating 400 nm polycarbonate membrane filters after a series of cyclic freezing and thawing steps in the range of −80° C. to 40° C. utilising a laboratory freezer and a heated water bath. This step aimed to increase the capture volume with the formed liposome. The LUVs were separated from any unencapsulated material by gel filtration using an Amersham Pharmacia AKAT gradient processing FPLC system complete with a 900-model monitor, lamp and detector (set at 280 nm), 920 model pump and incorporating a Frac 950 fraction collector interfaced to a Compaq Desk Pro Pentium III computer supporting Unicorn analytical software. The column used for the chromatograph was a K9-30 column packed with Sepharose CL 4B beads (45 um to 165 um) and fitted with two 25 um supporting filters. The column was packed using a RK16/26 packing reservoir and filled using a variable speed peristaltic pump. The particle size of the formed LUV liposomes was estimated on a Malvern Mastersizer-X-long bed particle analyser interfaced to an ACO Pentium II computer supporting Mastersizer Version 2.18 analytical software. A Bioline orbital shaker was used to incubate the yeast with washing, pelletising and density separations for cells and protoplasts undertaken on a Beckman J2-H2 centrifuge utilising a fixed head JA 20 rotor as well as a JS-13 swing bucket head rotor specifically for the separation of protoplast. Confirmation of protoplast viability was undertaken by C 14-sucrose uptake measured on a Packard 1600TR liquid scintillation analyser after pelletising the protoplasts on a Beckman 152 microfuge through a silicon oil gradient.

Confirmation of fluorescent endocytosis was undertaken on a Leica TCS-4D Confocal Microscope fitted with a Krypton-Argon mixed gas laser set at an excitation wavelength of 488 nm which was interfaced to a Dell Pentium 2 computer supporting Leica Scan-ware imaging software. Detailed cell imaging to identify the position of liposomes within the cytoplasm and plasma membrane undertaken on a Philips CM 100 Transmission Electron Microscope with images captured on a Gatan Dual Vision Digital camera.

Experimental Methodology:

Creation of Liposomes:

Large unilamellar vesicles (LUV) were prepared according to standard techniques with modifications. Prior to use in the trials all glassware was washed in a phosphate free detergent followed by immersion in 5M nitric acid and rinsed in Milli Q water to remove any trace of residual proteinaceous material, lipid or salt in an effort to avoid unwanted oxidation of the phospholipids or the possible insertion of any extraneous materials into the formed lipid bilayers which would change the charge or packing density of the created lipsomes.

Preparation of Standard Phospholipid Solutions:

Stock solutions of 20 mg/ml were prepared for phosphatidylcholine (PC), phosphatidylethanolamine (PE), dioleoylphosphatidylethanolamine (DOPE), 1,2-dioleoyl-sn-glycero-3-trimethylammonium-propane (DOTAP) and ergosteral (ERGO). They were prepared by dissolving 20 mg of each lipid in to 1 ml of a 2:1 (v/v) solution of chloroform and methanol which had been filtered through a 0.45 um Millipore filter and stored at 4° C. until use.

Only glass flasks, beakers, syringes and stainless steel needles were used in the preparation of these mixtures to avoid the possible migration of plastic monomers from the dispensing pipette tips or filters which could contaminate and alter again the packing configuration of the phospholipids micelles which could result in premature leakage of the entrapped materials upon storage of the liposome suspension. The formulation of each liposome varied with phospholipid composition, fatty acid composition, sterol content and surface charge.

Preparation of the Dried Lipid Film:

To a washed 100 ml Quick fit round bottom flask was added 5 ml of the 2:1 Chloroform-Methanol solution to ensure adequate dispersion of the phospholipids. A total of 250 μl of mixed lipids from the 20 mg/ml stock solutions was then added and gently mixed for 1 hour. The flasks were wrapped in aluminium foil to protect the lipids against oxidation by UV light and the headspace was nitrogen flushed for 5 minutes to remove oxygen from the headspace prior to attaching a flask to the Buchi rotary evaporator. The solvents were evaporated under vacuum for 90 minutes at 60 rpm in a water bath set at 37° C., which is above the phase transition temperature (Tm° C.) for the phosphatidylcholine component thereby ensuring all lipids were in the liquid crystalline phase allowing for a uniform dispersion. The evaporator was also covered in a black plastic film to further ensure a minimal passage of light. To ensure the removal of all solvents from the dried lipids, the flasks were finally freeze dried at −52° C. at 0.001 Mbar pressure for 2 hours then re-flushed with nitrogen, sealed and stored at −80° C. until required for re hydration.

Hydration of the Dried Lipid Film and the Encapsulation of a Fluorescent Dye:

The dried phospholipid films were hydrated in a two-stage process to avoid the precipitation or flocculation of the charged cationic lipid DOTAP which is sensitive to millimolar concentration of poly anions such as calcein, phosphate or EDTA as well as the presence of either monovalent or divalent cations in concentrations higher than five millimolar. The separation phenomenon of cationic lipids were observed during our work with the process investigated and corrected during our trials for this formula by hydration of the film initially in Milli Q water at 37° C. for 30 minutes to initiate the formation of a micelle complex. This was then followed by a 30 minute to 1-hour hydration with the desired encapslent in an appropriately buffered solution at pH 7.2 to induce a cationic sensitivity onto the liposome. Our initial trials used a 20 mM solution of Calcein in 10 mM of Trisma adjusted to pH 7.2 and then continued with a 10 mg/ml solution of Fluorescein isothiocyanate dextran FD 250S to ensured the passive movement of a smaller molecule across the plasma membrane of our formed protoplasts was avoided. It was identified in earlier trials that when the smaller molecular weight calcein was incubated alone with the formed protoplasts there was some passive migration of this molecule across the plasma membrane or our cells producing a false positive result. Substitution of calcein with the FITC 250 kDA dextran eliminated this situation and provided accurate data on liposomal induced endocytosis. The hydrated lipids were then cyclic frozen and thawed 3 times from −80° C. to 37° C. in an effort to increase the capture of volume of the fluorescent dye within the lipid bilayers while avoiding the break down of the FITC conjugated dextran. A brief bath sonication for 10 minutes was undertaken to finalise development of the MLVs while reducing any excessive aggregation between the micelles. Each solution was then extruded in an Avestin Lipo fast basic with stabiliser extruder.

Extrusion and Isolation of the Large Unilamellar Vesicles by Gel Filtration:

The Avestin lipofast basic extruder is a hand driven extrusion device with a capacity of 1 millilitre utilising 2 purpose built Hamilton gas tight syringes fitted to a pair of lour locked membrane supports that enclose a polycarbonate filter which is bound within a stainless steel housing. Prior to each use, the extruder components are washed in water than with a 2:1 Chloroform: Methanol solution to remove any residual lipid and rinsed again in Milli Q water and allowed to dry prior to assembly. Our experiment used a 400 nm disposable polycarbonate filter that was fitted to the membrane supports with tongs to avoid contamination with ancillary lipids. Later work identified that 200 nm filters may be more appropriate for an enhanced endocytotic entry of our liposomes across the cell wall based on transmission electron micrographs of large liposomes adhering and smaller liposomes passing into the cytoplasm of the Protoplasts. The drawback of this assumption is that the capture volume of the lipsome will also reduced. Upon assembly of the extruder, the syringes were individually filled with the 10 mM Trisma buffer at pH 7.2 and extruded through the polycarbonate filter into the opposing syringe and the contents discarded. This was repeated with the other syringe but undertaken in the opposite direction with the intention of pre wetting the filer pad for an easier passage of the lipid solution through the membrane as well as removing any remaining dye or lipid which may have been bound within the casing of the extruder.

The extruder and the flasks containing the MLVs were immersed in a water bath at 37° C. to preheat the apparatus and ensure the lipids were in a liquid crystalline state ensuring an easier passage through the extruder. The solutions were passed through the extruder in a forward and backward motion 23 times to achieve a uniform homogenous dispersion of LUVs. For each extrusion it was important to finish the procedure in the opposite syringe to avoid the re suspension of any trapped material which may be present on the filter during the first pass. The solutions, which became clearer on repeated extrusions, were filled into 2 ml pre sterilised eppendorf tubes which were covered in aluminium foil and stored at 4° C. prior to gel filtration.

The buffer used for the gel filtration was similar to that solution which suspended the protoplasts during the density gradient experiments and as such was therefore capable of supporting the protoplasts and liposomes during later incubations for endocytosis studies. The buffer consisted of a 1M sorbitol, 100 mM potassium chloride, 25 mM trisma base and 100 uM magnesium chloride adjusted to pH 7.2. Prior to use, the buffer was filtered and degassed under vacuum for 10 minutes and prepared in Milli Q water. After filtration, the buffer was also boiled to remove any remaining air and held in a jacketed bath at 37° C. to ensure the lipids were in a liquid phase during the filtration. The requirements for removing trapped air from the buffer prior to its use was to increase the degree of separation between the liposomes and the free dye by avoiding the compression of the soft Sepharose packing beads. Prior to use, the column was equilibrated with three volumes of buffer to remove any residual ethanol or sodium azide which was used as a preservative within the FPLC lines and the Sepharose packed column, while not in use the columns were stored in the refrigerator at 4° C. The separation protocol used a flow rate of 0.5 ml/min and two millilitres of liposome suspension were injected into the column and an elution efficiency of 95%. The process volume used was 79 mls and the process time was 197 minutes.

Creation and Confirmation of Competent Yeast Protoplasts:

Preparation of Culture Media and Incubation of Yeast Cells:

For the production of yeast protoplasts, a freeze-dried culture of Saccharomyces cerevisiae (YNN 281) was recussitated and then propagated for 20 hours at 30° C. in a temperature controlled shaker set at 120 rpm and grown in a sterilised YEPD media containing 1.5% (w/v) yeast extract, 2.0% (w/v) bacterial peptone and 2.0% (w/v) dextrose adjusted to pH 6.5. After incubation, the yeast cells were washed twice in an osmotically stabilised solution containing 0.65M potassium chloride, 25 mm trisma base and 100 um magnesium chloride adjusted to ph 6.5. Centrifugation of the harvested cells were undertaken at 4° C. in a Beckman J2 H2 centrigue at 10,000 rpm for 15 minutes.

It has been identified that the porosity and lipid content of the yeast plasma membrane can be altered for a facilitated endocytotic transport of higher molecular weight compounds if the conditions for time, temperature or pH are manipulated or the solutes in the supporting media were modified to enhance lipid membrane growth.

It has been shown that if the media containing yeast cells were to contain 0.1% (v/v) oleic acid or up to 1.0% (v/v) Tween 80 and the cells were incubated at a temperature below 20° C. combined with the addition of an organic solvents in the growth media such as ethanol, chloroform to toluene or contained either 30 mM of trisma, 1% (w/v) sodium chloride or grown on a lactose based substrate at a reduced level of orthophosphate at a pH of 5.5, the total lipid content of the cell could be increased by up to 27% together with an increase in the permeability of the yeast plasma membrane for an increased endocytotic passage of the lipsome. Any of these variables may be evaluated to improve the rate at which the pH sensitive, cationic lipsomes could penetrate the plasma membrane of a yeast protoplast. The yield of cells derived from our YEPD trial media after 20 hours incubation was determined by hemocytomer to be 5.10⁷ cell/ml.

The Preparation of an Enzyme Solution and Density Gradients for the Isolation of Yeast Protoplasts:

For the digestion of our yeast cell walls an enzyme digest was prepared containing 2 mg/ml of Lyticase and 1 mg/ml of snail gut juice (β-Glucuronidase from Helix pomatia) in a solution of 0.65M potassium chloride, 25 mM trisma base and 100 uM magnesium chloride adjusted to pH 6.5. This is the same solution that was used for the washing and re suspension of the harvested yeast cells. The protoplasts were prepared from a 4 ml washed cell suspension with 1 ml of enzyme preparation added. Incubation was undertaken in a sealed conical flask and reacted for 3 hours at 37° C. on a temperature controlled rotary shaker set at 120 rpm to produce our viable protoplasts.

A five-tier density gradient was then constructed in Falcon tubes to isolate protoplasts from intact cells and cell wall remnents in the range of 1.04 g/l to 1.10 g/l. Each solution comprised sorbital from 0.65M to 1M in concentration, 25 mm of trisma base, 100 um of magnesium chloride and either 5 or 10% Ficoll in the densest fractions. Each solution was stabilised to pH 7.2 and a total of 5 ml of each solution was layered into the falcon tubes beginning with the densest fraction and finishing with the yeast—enzyme digest. The tubes were then capped and place in the Beckman centrifuge and spun for 15 minutes at 1000 rpm and 4 C to isolate the protoplasts.

Confirmation of Yeast Cell Viability by C14 Sucrose Adsorption:

To ensure the competent nature of the formed protoplast to undertake endocytosis, the yeast cells were grown on a modified YEPD media in which the 2% (w/v) dextrose was replaced by 4% (w/v) sucrose as the carbon source to facilitate a mechanism for the absorption and transport of the sucrose C14 by the yeast cells. Protoplasts were formed as described above and isolated again utilising a sorbitol based density gradient. Silicon oil isolation of protoplasts is a convenient method for separating radio labelled cells from a sugar gradient solution. In this procedure cells are incubated in a radioactive solution for a specified period of up to 1 hour as seen in our trials and then spun through silicon oil at a specific density to separate them from scintillation counting. Seven micro tubes (400 ul) were prepared for pelletising the protoplasts with each containing 10 ul of Fluorinert FC 77 as a capture solution and 100 ul of Silicon Oil AR 200 (Density of 1.05 g/l) as the media to be used for the separation of sucrose C14 protoplasts. In the experiment 40 ul of a 1M sucrose (made in a 50 ml volumetric flask) was used to which was added 2 ul of the sucrose C14 stock. Then 1.2 ml of the protoplast suspension was placed into a 1.5 ml eppendoff tube and 12 ul of the 1M sucrose C14 solution was placed in the cap. All work was undertaken in a specified radio laboratory within an insulated fume cupboard. The incubation time started when the cap was sealed onto the eppendoff tube. Next, 100 ul aliquots of the protoplasts were taken at 10 minute intervals and placed sequentially into a series of the prepared 400 ul tubes containing the silicon oil gradients and spun at 7000 rpm for 10 second. The tubes were removed at each time interval and cut through the upper silicon oil fraction to capture the formed protoplast pellet and resuspended in 100 ul of Milli Q water in a scintillation tube containing 3 ml of scintillation fluid. As a standard, 100 ul volumes of the 1M sucrose containing the sucrose C14 were the silicon oil and counted to avoid quenching correction of the protoplast extract.

A graph of time vs. counts, recorded as nmoles of absorbed sucrose was then prepared to confirm the uptake of our sucrose isotope by the competent protoplasts.

Endocytosis Studies

Incubation of Formed Protoplasts with Dye Bound Liposomes:

A 0.8 ml aliquot of protoplast suspension was dispensed through a large orifice pipette tip (ensuring minimal disturbance of the fragile protoplast) into a sterilised eppendoff tube to which was added 0.8 ml of the liposome suspension obtained from the gel filtration. Both solutions were of equivalent densities to ensure the stability of the protoplast and gently mixed prior to incubation. The tubes were individually wrapped in foil and embedded on their side in a sheet of Styrofoam which was also wrapped in foil to ensure the tubes would remain in place and then immersed into a water bath at 37° C. for the 90 minute incubation.

Confocal Microscope Studies Identifying Liposomal Fusion and Fluorescent Endocytosis:

A 20 ul sample of the incubated suspension was applied to a microscope slide with the edges of the cover slip sealed with an acrylic resin to inhibit drying out of the sample. The slides once made were stored in the dark to reduce the risk of a loss in fluorescence of the FITC conjugate and viewed immediately under phase contrast and fluorescence using the confocal microscope.

Transmission Electron Microscope Studies Confirming Liposomal Coalescence and Cellular Endocytosis:

The technique used for preparing sections of block-mounted cells for image analysis required 0.5 ml of the protoplast-liposome suspension to be fixed with 50 ul of 1.25% (v/v) glutaraldehyde and 1.0% (w/v) paraformaldehyde in a 0.2M cacodyl ate buffer at pH 7.2 for 1 hour. The fixed suspension was then missed with a similar volume of 5.0% agarose and placed at 4° C. for 20 minutes to set. Small cubes of the mixture (approximately 2 mm3) were cut and fixed for a further 14 hours at 4° C. Samples were then post fixed with 1.0% (w/v0 osmium tetroxide, stained en bloc with 2.0% (w/v) uranyl acetate, dehydrated through a graded ethanol series and embedded in Epon/Araldite resin.

Ultra thin sections were cut on a Leica ultramicrotome and collected on copper/palladium grids. Sections were stained with 4.0% (w/v) uranyl acetate and Reynolds lead acetate and viewed with a Philips CM 100 Transmission Electron Microscope with images captured on a Gatan Dual Vision digital camera.

Results and Discussion:

The Structure of a Yeast Cell Wall and the Requirement for Producing a Liposome of Similar Lipid Composition.

Liposomes are defined as structures of one or more concentric spheres of lipid bilayer. Each layer is separated by an aqueous space of capable of encapsulating either water or lipid soluble materials in or between each layer. The inner membrane of a yeast cell wall is also composed of a similar structure of lipid bilayers.

The study begins with the incorporation of two phospholipids, PE and PC, into the primary matrix of a liposome in a ratio similar to that found in a yeast cell wall during its exponential growth. The inclusion of a charged amphiphile, DOTAP will aim to incite initial fusion to the surface proteins found on the yeast plasma membrane and the stabilising agent, ergosterol will further aim to facilitate an easier lipid exchange given its presence in the cell wall and unique proximity to the Sphingomyelin rafts again present in the yeast cell wall. Additional compounds have also been built in to the liposome structure to facilitate a controlled release of the encapsulated material and are based on phosphatidylethanolamine and its derivative DOPE which contain a unique high degree of unsaturated fatty acids at a chain length and compositional ratio similar to that found in the yeast phospholipid membrane during growth. It has been identified that the phospholipids derived from Soy have a similar fatty acid composition to that present in the yeast cell wall and will be used exclusively for our work.

To prepare a liposomal for endocytosis requires an understanding of the composition of a yeast plasma membrane and primarily its fatty acid composition. This was a difficult area to research as a defined lipid composition for Saccharomyces cerevisiae was not only broadly defined but varied considerably between authors as a result of the components used in the growth media, the conditions used for incubation as well as the methods used for the extraction and analysis of the individual lipid types. Some variations were also identified within different species of yeast at different stages of their development but this was not considered for our current work.

The purpose of investigating the composition of a yeast cell membrane was primarily to identify the phosopholipid types seen on the outer lipid bilayer during a time when the cell wall is maturing and is relatively flexible and capable of allowing lipid exchange during either mitosis or endocytosis. Identifying the charge potential on the surface of the cell as well as the chain length and degree of unsaturation of the fatty acids components of these phospholipids of the outer membrane is vital if a liposome is to be created which replicates this structure allowing liposomal endocytosis. The two membranes upon coalescence aim to interchange their lipids thereby weakening both membrane structures, which aids in the release of compounds from the liposome into a softened endosomal compartment for migration into the cell cytoplasm.

Phospholipids form smectic mesophases that undergo a characteristic gel-liquid phase transition. Vesicles composed of phospholipids that are at a temperatures below the phase transition temperature (Tm° C.) for that lipid are considered “solid” while those above are considered ‘fluid’ crystalline. The phase transition temperature is a function of acyl chain length and (Tm° C.) increases approximately 14° C.-17° C. with the addition of every two methyl units. The inclusion of unsaturated acyl chains, mixed chain lengths or bulky side groups like cyclic propane rings will each produce a considerable change and in the case of side chains a decrease in the phase transition temperature.

The head groups of the phospholipids can also have an influence on the phase transition temperature as well as the lipid packing density as a function of the size of the molecule attached to the phosphate moiety as well as the charge applied when influenced by the interaction with specific cations or H+ ions at a reduced pH.

As mentioned the source material of phospholipid can contributes greatly to a change in the phase transition temperature of each lipid variety because of the presence of unsaturated double and triple bonds in the respective acyl chains. Phosphatidycholine derived from egg is more prone to oxidation compared to phosphatidycholine derived from soy and highlights the need to choose the appropriate raw material to create a stable liposome that has an appropriate release mechanism based on the target cells fatty acid composition that allows it to facilitate encapsulent release based on an increased temperature and a differential pH on either side of the adjoining membranes during cellular incubation with a liposome.

TABLE 1

| | | | | | Phosphatidyl- | | Phosphatidyl- |
|---|---|---|---|---|---|---|---|
| | Phosphatidyl- | | Phosphatidyl- | | ethanol- | | ethanol- |
| Egg | choline | Soy | choline | Egg | amine | Soy | amine |
| 16:0 | 67.1% | 16:0 | 15.8% | 16:0 | 37.9% | 16:0 | 24.3% |
| 18:0 | 23.2% | 18:0 | 4.6% | 18:0 | 52.7% | 18:0 | 2.2% |
| 18:1 | 5.6% | 18:1 | 13.3% | 18:1 | 7.7% | 18:1 | 6.7% |
| | | 18:2 | 60.9% | | | 18:2 | 60.3% |
| | | 18:3 | 5.3% | | | 18:3 | 6.2% |

Table 1 provides a comparison between the fatty acids in PC and PE from different sources. The presence of Palmitic acid and Stearic acids or a mixture of acyl groups in egg phosphatidylcholine can affect the phase transition temperature of the lipid considerably. Using phosphatidylcholine as an example the Tm° C. for 16:0 is 41° and 18:0 is 55° C. with a mixture of 16:0 and 18:0 fatty acids between 44° C. and 49° C. compared to 12° C. for 18:1 and −53° C. for 18:2. The above table highlights that phosphatidylcholine and phosphatidylethanolamine particularly derived from soy will generally be in a liquid crystalline phase given their level of unsaturation and higher acyl chain length and be therefore easier to disperse at lower temperatures and be more likely to be involved in lipid exchange with a yeast cell membrane given their similar composition and while being less prone to oxidation.

Phosphatidylethanolamine was used with DOPE in the release mechanism for our liposomes, it has a smaller head group compared to Phosphatidylcholine and is inherently cone shaped and prone to forming hexagonal shaped, inverted micelles in solution particularly at a lower pH. The presence of Phosphatidylethanolamine and particularly Dioleoylphosphatidylethanolamine, (DOPE) with two unsaturated Oleic groups provides for a higher membrane curvature within the liposome that leads to a faster facilitated release of the encapsulated material and as such was incorporated to operate as the trigger release compound within the formula.

Soy phospholipids was chosen for our evaluation because they had the most similar structure and fatty acid composition compared to the Saccharomyces cerevisiae plasma membrane structure and using PE and DOPE provide for a quicker facilitated release due to the presence and configuration of the unsaturated groups with the acyl chain that ensured the structure remained in a liquid crystalline phase upon hydration and permitted a facilitated quick release upon an increase in temperature.

Ergosterol was trialed because it represented a 10 molar % component of the yeast cell wall and while sterols are generally associated in drug delivery with reducing membrane permeability and liposomal leakage, its presence in our formulation could only be assumed to enhance the endocytotic delivery of our pH sensitive liposomes across the plasma membrane of a viable yeast protoplast given the quick lipid exchange between the membranes we have now fused and the larger sterol providing enhanced pores and gaps near the sphingomyeline raft areas to destabilise the membranes structure.

The wall of Saccharomyces cerevisiae accounts for between 15% and 25% of the dry weight of the cell wall, and is between 100 nm to 20 nm in diameter and its function is protect the cytoplasmic components bound with the wall. The composition and structure of the yeast cell wall consists of an 80% to 90% mixture of carbohydrates and proteins with 7% to 14% total lipid.

The main structural components forming the outer region of the yeast cell wall are the polysaccharides and are based on protein conjugates comprising Mannan-proteins (˜25%) and three types of Glucan-linked proteins that are differentiated based of their solubility. These Glyco-proteins are either an alkali insoluble and acetic acid insoluble β-(1-3) linked glucans (37%), an alkali soluble β-(1-6) linked glucan and an alkali soluble β-(1-3) linked glucan (34% combined) in which the latter can be anchored to the plasma membrane for structural stability with a minor proportion of Chitin (4%) comprising the lipid network of the membrane.

The inner plasma membrane is approximately 7.5 nm wide and consists of a 50:50 mixture of polar lipids and proteins comprising Phosphatidylcholine (42-49%), Phosphatidylethanolamine (20-30%), Phosphatidyl inositol (10-20%) and phosphatidylserine (9-10%) with a smaller percentage of Sphingolipids and trace quantities of phosphatidic acid, Phosphatidyl glycerol and free fatty acids, the latter of which may be artefacts that are produced as a function of the method of extraction. The Sphingolipids are long chain unsaturated amino alcohols having a similar structure to phospholipids with one of the hydrocarbon chains a Sphingosine (long chain unsaturated amino alcohol). Their functions in cell membranes are as signal transducers and cation mediators across the lipid bilayer. Enzymes form the majority of protein based derivatives within the cell wall and are present within and just below the plasma membrane associated with the transport of ions and solutes also across the lipid bilayer.

The lipids present within the plasma membrane represent the majority of the total lipids found within the cell on a dry weight basis with half of these present as phospholipids. Phospholipids are amphiphilic molecules with both polar and non-polar ends associated with their structure. The polar head groups onto which an organic alcohol is esterified to a phosphate group will generally possess a negative charge but may be zwitterionic as is the case with the head group of phosphatidylcholine as there is a positive charge on the nitrogen atom at an acidic pH. The size and degree of charge upon the head groups will affect the packing density of the lipids within the bilayers and as such will affect the permeability of both a yeast cell wall and a newly created liposome. The non-polar end is comprised of long chain hydrocarbons or “Fatty Acids” which can be methylated or contain double bonds.

The main fatty acid acids make up the phospholipids found within the yeast plasma membrane of Saccharomyces cerevisiae are Palmitic [C 16:0˜12], Stearic [C 18:0˜5.0%], Palmitoleic [C 16:1˜44%] and Oleic [C 18:1˜35%]. The unsaturated fatty acids represent approximately 80% of the total acyl groups controlling the ethanol tolerance and stress of the cell. Sterols represent approximately 10% of the dry weight of the plasma membrane with Ergosterol representing almost 90% of the total sterol content.

The lipids of the bilayer within plasma membrane are asymmetrically disposed with phosphatidylethanolamine, phosphatidylserine and phosphatidylinositol located primarily within the inner bilayer and Phosphatidyl choline and sphingolipids presently mainly in the outer layer. The lipids in the membrane can also during the development of the cell, freely diffuse within the plane of the membrane and undergo a series of rotational and transverse motions referred to as “FLIP FLOP” actions, particularly between PC, PE and PS lipids during the early growth phase and later apoptosis, giving rise to a compositional change on the surface of the bilayer with a further change in its charge potential and packing density.

It was therefore decided to produce liposomes using the following formulas based on soy phosphilipids which contained a similar chain length and degree of unsaturation to that found in a yeast plasma membrane to examine the possibility of cellular endocytosis. Given the complex nature of the yeast cell wall it was decided also that yeast protoplasts would be a preferred platform for studying liposomal fusion and endocytosis between the two membranes without the interference of ancillary carbohydrates and proteins.

The Trials that were Undertaken Comprised the Following Phospholipid Combinations:

All trials were undertaken in duplicate using a unencapsulated FITC conjugated dextran as a negative control to confirm non-endocytosis transport by an unencapsulated conjugate.

| | | | |
|---|---|---|---|
| 1. | Phosphatidylcholine | 50 | molar % (125 ul) |
| | DOPE | 40 | molar % (100 ul) |
| | DOTAP | 10 | molar % (25 ul) |
| 2. | Phosphatidylcholine | 50 | molar % (125 ul) |
| | DOPE | 30 | molar % (75 ul) |
| | DOTAP | 20 | molar % (50 ul) |
| 3. | Phosphatidylcholine | 40 | molar % (100 ul) |
| | DOPE | 40 | molar % (100 ul) |
| | DOTAP | 20 | molar % (50 ul) |
| 4. | Phosphatidylcholine | 50 | molar % (125 ul) |
| | DOPE | 30 | molar % (100 ul) |
| | DOTAP | 10 | molar % (25 ul) |
| | Ergosterol | 10 | molar % (25 ul) |
| 5. | Phosphatidylcholine | 40 | molar % (100 ul) |
| | DOPE | 30 | molar % (75 ul) |
| | DOTAP | 20 | molar % (50 ul) |
| | Ergosterol | 10 | molar % (25 ul) |
| 6. | Phosphatidylcholine | 100 | molar % (250 ul) |
| 7. | Phosphatidylcholine | 50 | molar % (125 ul) |
| | Phosphatidylethanolamine | 50 | molar % (125 ul) |
| 8. | Phosphatidylcholine | 50 | molar % (125 ul) |
| | Phosphatidylethanolamine | 40 | molar % (100 ul) |
| | DOTAP | 10 | molar % (25 ul) |
| 9. | Phosphatidylcholine | 50 | molar % (125 ul) |
| | Phosphatidylethanolamine | 30 | molar % (75 ul) |
| | DOTAP | 20 | molar % (50 ul) |
| 10. | Phosphatidylcholine | 40 | molar % (100 ul) |
| | Phosphatidylethanolamine | 40 | molar % (100 ul) |
| | DOTAP | 20 | molar % (50 ul) |
| 11. | Phosphatidylcholine | 50 | molar % (125 ul) |
| | Phosphatidylethanolamine | 30 | molar % (100 ul) |
| | DOTAP | 10 | molar % (25 ul) |
| | Ergosterol | 10 | molar % (25 ul) |
| 12. | Phosphatidylcholine | 40 | molar % (100 ul) |
| | Phosphatidylethanolamine | 30 | molar % (100 ul) |
| | DOTAP | 20 | molar % (50 ul) |
| | Ergosterol | 10 | molar % (25 ul) |

The understanding of yeast lipid chemistry has highlighted a number of issues for the requirements of phospholipids to be used in our trials for the production of liposomes. The methods employed to produce and purify our LUVs as well as the requirements for later endocytosis and controlled release into a cell will be discussed below.

Methods for the Manufacture, Sizing and Purification of Liposomes:

There are three major categories of Liposomes, Multilamellar Vesicles which contain 2 or more concentric lamellae arranged in an onion skin configuration and can range in size from 1 um to <100 um, Large Unilamellar Vesicles which have a single bilayer and size distribution in the range of 200 nm to 1 um and Small Unilamellar Vesicles which are less than 200 nm in size. For our work, we have chosen the Large Unilamellar Vesicles (LUV) because Multi Lamellar vesicles were to large to cross the yeast plasma membrane and Small Unilamellar Vesicles were too small and unable to trap significant quantities of our FITC Dextran or later enzyme.

Large Unilamellar Vesicles can be made by a variety of methods after initial hydration of the lipid film to form MLV, Probe or bath sonication is a time consuming process that precludes liposomes of unequal size and capture potential and in the case of probe sonication produces only a small volume of suspension which needs to be cooled in ice water to remove the heat generated from the tip of the probe. This process is not scalable or adaptable to the encapsulation of enzymes as the heat generated and the requirement to remove titanium fragments from the liposome suspension would result in the inactivation the enzymes to be encapsulated.

A dehydration-rehydration procedure can also be adopted and is scalable to 100 lts volumes but is also variable in the capture potential. Reverse Phase solvent evaporation, Ethanol Injection, detergent dialysis, can also be applied but the drawback of each of these protocols while currently used by many researchers, exposures our enzyme to either organic solvents or detergents which will significantly reduce its activity. The detergent removal procedure particularly requires exhaustive dialysis to remove the surfactants and would be prohibitively expensive for the food industry.

Micro fluidisation has also be used to prepare vesicles as can a French Press, but again enzyme denaturation by shear forces would make both procedures unacceptable.

In the study we used solvent evaporation for the lipids and a gentle freeze thaw and extrusion protocol for the production of LUVs because it allowed for a high capture volume of the fluorescent dye and permitted the easier incorporation of a cationic lipid and would be mindful when enzymes replaced the fluorescent dye. The only problem that was encountered was the precipitation of the cationic transfection reagent DOTAP when the films were rehydrated in a one step buffered salt protocol.

A series of trials was conducted to understand the mechanism for this precipitation and identified the components in the hydration solution that caused the flocculation.

Each trial used a 50 molar % Phosphatidylcholine (PC), 30 molar % dioleoylphosphatidylethanolamine (DOPE) and 20 molar % 1,2-dioeoylosy-3-(trimethylammonio) propane (DOTAP), prepared as a 3 mg lipid sample which was dissolved in a 2:1 Chloroform/Methanol solution. The charge affect of Calcein was also evaluated with the encapsulation of 5 mM, 10 mM and 25 mM solutions prepared in Milli Q water and directly added to the dried films. Flocculation occurred with all three samples upon direct contact and it was decided that a 10 mM Calcein solution would then be used for all remaining trials to determine the effect of a charged fluorophore.

The hydration solution for the trials consisted of mixtures containing 25 mM of Trisma base with either 5 mM or 50 mM of Calcium Chloride and 5 mM or 50 mM of Magnesium chloride adjusted to pH 7.2. Twelve trials were undertaken with precipitation occurring in all instances when the lipid film was hydrated in a one step process and at a higher solute concentration.

TABLE 2
Trials undertaken to determine the factors that caused
precipitation of a DOTAP containing lipid film.

| | |
|---|---|
| 1. | Hydration of the dried lipid film in Milli Q water alone. |
| 2. | Hydration of the dried lipid film in Milli Q water with a second a second |
| | addition of a 10 mM Calcein solution. |
| 3. | Hydration of the dried lipid film in a 10 mM Calcein solution. |
| 4. | Hydration of the dried lipid film in a 25 mm Trisma Solution adjusted to |
| | pH 7.2. |
| 5. | Hydration of the dried lipid film in a Trisma Solution adjusted to pH 7.2 |
| | with a second addition of Calcein. |
| 6. | Hydration of the dried lipid film in a 10 mM Calcein solution containing |
| | Trisma adjusted to pH 7.2. |
| 7. | Hydration of the dried lipid film in a 5 mM Calcium Chloride solution. |
| 8. | Hydration of the dried lipid film in a 50 mM Calcium Chloride solution. |
| 9. | Hydration of the dried lipid film in a 5 mM Magnesium Chloride solution. |
| 10. | Hydration of the dried lipid film in 50 mM Magnesium Chloride solution. |
| 11. | Hydration of the dried lipid film in a solution containing 5 mM Magnesium |
| | Chloride, 5 mM Magnesium Chloride, 10 mM Calcein and 25 mM Trisma. |
| 12. | Hydration of the dried lipid film in Milli Q water with the second addition |
| | of a solution containing 5 mM Magnesium Chloride, 5 mM Magnesium Chloride, 10 mM |
| | Calcein and 25 mM Trisma. |

All films were hydrated in a water bath at 37° C. and held for 30 minutes after each solution addition.

Solutions 1, 2, 4, 7, 9 did not show any signs of precipitation, solutions 5 and 12 showed minor signs of precipitation with all other variants precipitating upon mixing. It can be concluded that at a higher concentration of salts in a solution the cationic lipid DOTAP precipitated. The FITC conjugated dextran did not precipitate the cationic lipid in our subsequent trials when the liposome film was the first hydrated in water and no other compound were used in the hydration solution for the remaining endocytosis trials.

### The Freeze-Thaw and Extrusion Protocol Using Polycarbonate Membranes to Produce pH Sensitive Cationic Large Unilamellar Vesicles:

The inclusion of a preliminary freeze-thaw step prior to extrusion, provides for and increase in the capture volume within the core of the formed LUV as confirmed also by our TEM observations. The principle of freezing and thawing causes a rupture and re-fusion of the liposome during which time the encapsulated solute equilibrates in the deeper cavities of the liposomal core. The liposomes upon freezing and thawing can also aggregate however it was seen that a brief sonication step prior to extrusion could disassociate the cluster and aid in the formation of LUVs.

In experiments with Calcein, up to 6 freeze thaw cycles were used to increase the capture volume however in the case of the FITC conjugated Dextran, only 3 cycles were preformed in an effort to avoid breaking off the fluorescein compound which if free could provide a false positive result as was seen in the endocytosis studies with Calcein.

The size of the formed liposomes was tailored to <400 nm with the action of the extruder reducing the risk of denaturing enzymes or conjugates as well as fouling the membranes. The filter used were a 400 mm filter although a 200 nm filter was later evaluated to increase the percentage of small LUV for easier migration across the lipid membrane of the cell. The polycarbonate membranes used for the extrusion are produced by a combination laser and chemical etching process, which aims to produce straight-sided pore holes of exact diameter.

It was also found that during our trials that there was a necessity to hydrate and extrude our lipids above the phase transition temperature of the highest temperature lipid to avoid tearing of the polycarbonate filter pads during extrusion.

The extrusion procedure produced LUVs of a uniform, defined size as determined by the Malvern Mastersizer S, and confirmed by Transmission Electron Micrographs. The Histogram identifies that the sample applied represented 57.4% of liposome were less than 400 nm with an average range size of 360 nm.

The process of gel filtration was used to fractionate the formed liposomes from the solution containing the unencapsulated dye. The successful separation of the cationic liposomes required an understanding of the principles of separation based on a differential size of the materials and the capability to pack an efficient gel filtration column. Sepharose CL 4B was the packing material used for the separation and is a bead-formed agarose which is derived from agar and cross-linked by reaction with 2,3 di-bromopropanol under alkaline conditions. The cross-linking effect of this material as in the case of cross-linked starches provides the agarose gel with a greater thermal and chemical stability over a wide pH range.

Sepharose CL 4B chosen because it has a smaller bead size within its bottom range which facilitates a faster movement of larger molecular weight components. It has an optimum separation range between 70×10²-20×10² and a bead size in the range of 45 to 165 um.

The filters for the column were chosen specifically to be 25 um to ensure the smaller size beads within the column would remain and the liposomes would separated and not become lodged on to the top of the column filter restricting the flow and hindering the separation as seen when early columns were used for our trials. The conventional filters were identified to be 2 um in size causing fouling and restricted the entry of the large liposomes into the column, prohibiting their isolation.

The separation buffer used in the trials was chosen because it could osmotically support the formed protoplasts when combined with the later liposomes for endocytosis studies and identified during the gradient separations for the formed protoplasts. Separation of liposomes from gel filtration was identified by two eluent peaks on the chromotograph in with the milky suspension containing the liposomes exiting the column first as a function of its molecular size and confirming by TEM images, negatively staining on carbon coated grids with 2.0% (w/v) solutions of sodium phosphotungstate adjusted to pH 7.0

The Formation and Identification of Competent Yeast Cell Protoplasts:

Protoplasts were prepared based on the methods as follows. A three-hour yeast cell and enzyme incubation was undertaken with post centrifugation through a defined density gradient to isolate the protoplasts performed at 1000 rpm for 15 minutes at 4° C. Protoplasts were found primarily in the third layer of the falcon tube with the solution having a density of 1.07 g/l. This buffer concentration was then used as the separation solution for all gel filtration experiments. Protoplasts were identified by phase contract and TEM microscopy with remnants of cell wall present and captured. It was observed during the confocal and TEM microscopy that remnants of the cell wall containing the negatively charged proteins formed clusters with the DOTAP liposomes particularly at the 20 molar % concentration.

The addition of a two-component enzyme system using Lyticase and Snail Gut Juice, (β-Glucuronidase from Helix pomatia) target the hydrolysis of β-(1-3) glucans and the cysteine bonds within proteins of the cell wall to effectively hydrolyse both carbohydrates and proteins producing the protoplasts. Trials were undertaken at a constant temperature of 37° C. and at a pH of 7.2 because we had identified pH not to be a critical parameter and all prior hydrations and purification steps were conducted at this temperature. The only variable that was reviewed was the incubation time, which varied from 30 minutes to 90 minutes. It was decided that 90 minutes would be the most appropriate time to achieve the highest yield of viable protoplasts.

To ensure the uptake of our liposomes by endocytosis, viable protoplasts had to be determined and the use of a radio labelled isotope of sucrose was adopted with the uptake of this metabolite recorded over a one-hour period. Cells were grown in a sucrose media to facilitate the desired cellular transport mechanism as this was the only isotope available for the study.

Standards were prepared and measured for background scintillation without the added isotope and 100 ul of a 10 mM sucrose standard continuing the isotope was measured to obtain counts for a 1 umole solution of sucrose. The scintillation result showed the 1 umole of sucrose provided approximately 7000 counts, therefore 1 count was calculated to be 1×10⁻⁶/7000 or 144×10⁻¹² moles. Background counts for the non-isotope sucrose solution was 9 counts. The result from this experiment showed a linear and progressive uptake of the sucrose isotope as identified in the table below highlighting the competent and viable nature of the formed protoplasts.

TABLE 3
Time and scintillation counts for the determination
of I-Sucrose 14 C uptake by yeast protoplasts

| | | Scintillation count - | I-Sucrose 14 C |
|---|---|---|---|
| Time (mins) | Scintillation count | background count | uptake (nmoles) |
| 1 | 22 | 13 | 1.87 |
| 10 | 55 | 46 | 6.62 |
| 20 | 102 | 93 | 13.4 |
| 30 | 133 | 124 | 17.9 |
| 40 | 194 | 185 | 26.6 |
| 50 | 258 | 249 | 35.9 |
| 60 | 404 | 395 | 56.9 |

Incubation and Endocytosis Trials for Cationic Liposomes and Viable Yeast Protoplasts:

Movement of solutes across the membrane can be undertaken in a number of ways, depending on the size and the charge of the solutes by the process of either diffusion or absorption. Fusion, inter membrane transfer and exchange of lipids (between liposomes and cell membranes) results in the facilitated uptake of the large molecular compounds by cells with their delivery into early endosomes. The uptake process occurs by the action of either phagocytosis but usually endocytosis and requires solutes to be encapsulation in a micelle with an opposite surface charge to that of the cell but also by of similar chemistry to the lipids in the target membrane.

During endocytosis, an initial coalescence of liposomes with a cells plasma membrane occurs when the cells surface proteins or enzymes that are negatively charged bind to a positive charge that has been applied onto the liposome with the further possible addition of stabilising cations, antibodies or Proplylene Glycol to ensure protection and correct orientation of the functionally active enzymes bound to the cells surface.

Once fixed to the surface of the cell, the liposome becomes enveloped by the plasma membrane. It is important to recognise the predominant positioning of the Phosphatidylcholine on the outer bilayer of the cell membrane during the growth of the yeast providing for fusion and lipid exchange with the cationic PC based liposome.

Once brought into the cell, the liposome resides in an early endosome. The action of a reduced pH environment within the cell of between 5.6 and 5.9 combined with an elevated incubation temperature of 37° C. encourages the exchange of lipids between the 2 membranes resulting in a weakening of the lipsome and endosomal structure.

The requirement for a similarity in membrane composition is highlighted by the formula containing ergosterol at 10 molar %. At an elevated temperature, the fluidity of the LUV and the alteration in the packing density caused by the ergosterol aided in the facilitated the release of the conjugated dextran into the cell protoplast. The main release mechanism as identified in our work was the Phosphatidylethanolamine and DOPE components which contained Oleic acid as identified by the fluorescence seen within the cells with the use of formulas 4, 5, 11 and 12.

TABLE 4
A summary of the endocytosis trials with the various
liposomal formulas containing FITC Dextran

| | | | |
|---|---|---|---|
| 1 | PC | 50 molar % | Some fusion with the protoplast was seen under |
| | DOPE | 40 molar % | confocal, endocytosis was not observed |
| | DOTAP | 10 molar % | |
| 2 | PC | 50 molar % | Additional cell fusion was observed between the |
| | DOPE | 30 molar % | two membranes with clumping of cell remains |
| | DOTAP | 20 molar % | around the cationic liposomes also observed, there |
| | | | was no indication of endocytosis |
| 3 | PC | 40 molar % | Again, a higher degree of fusion was seen with the |
| | DOPE | 40 molar % | liposomes found around the outside walls of the |
| | DOTAP | 20 molar % | yeast protoplasts. A higher degree of clumping was |
| | | | seen, no indication of endocytosis. |
| 4 | PC | 50 molar % | This was the first indication of endocytosed |
| | DOPE | 30 molar % | liposomes as identified by fluorescing cells as |
| | DOTAP | 10 molar % | confirmed by confocal phase contrast and |
| | ERGO | 10 molar % | fluorescence overlay and Transmission Electron |
| | | | Microscopy showing liposomes in the cell |
| | | | cytoplasm. |
| 5 | PC | 40 molar % | There was signs of endocytosis in the confocal |
| | DOPE | 30 molar % | images, not as distinct as in trial 4 but contained |
| | DOTAP | 20 molar % | more fusion between the liposomes and protoplasts. |
| | ERGO | 10 molar % | Additional time may enhance the delivery. It |
| | | | appears that Ergosterol and a higher PC content may |
| | | | be an advantage. |
| 6 | PC | 100 molar % | No fusion or endocytosis seen. |
| 7 | PCPE | 50 molar % | No fusion or endocytosis seen. |
| | | 50 molar % | |
| 8 | PC | 50 molar % | Again, some fusion seen between the cationic |
| | PE | 40 molar % | liposomes and the cell protoplasts, no endocytosis. |
| | DOTAP | 10 molar % | |
| 9 | PC | 50 molar % | Additional clumping seen between the cationic |
| | PE | 30 molar % | liposomes and the free cell wall material with |
| | DOTAP | 20 molar % | liposomal fusion observed |
| 10 | PC | 40 molar % | Fusion was again observed with clumping of cells |
| | PE | 40 molar % | and cell remnants around the cationic liposomes |
| | DOTAP | 20 molar % | |
| 11 | PC | 50 molar % | Some fluorescence was seen inside the cell but was |
| | PE | 30 molar % | not as pronounced as the liposomes containing |
| | DOTAP | 10 molar % | DOPE. the release mechanism appears to require |
| | ERGO | 10 molar % | DOPE or the addition of Oleic acid into the |
| | | | liposomal matrix with an additional cationic charge |
| | | | to compensate for the negative charge on Oleic |
| | | | Acid. |
| 12 | PC | 40 molar % | Enhanced liposomal fusion was seen with the slight |
| | PE | 30 molar % | appearance of glowing cells. DOTAP appears to |
| | DOTAP | 20 molar % | contribute to increased fusion with the outer cell |
| | ERGO | 10 molar % | membrane of the protoplast but the presence of a |
| | | | higher PC with Ergosterol component appears to |
| | | | enhance the progress of endocytosis. |

| | | |
|---|---|---|
| 13 | FITC Dextran | No fusion or endocytosis |

All trials were conducted in duplicate with a negative control evaluating the endocytosis of unencapsulated Dextran

Confocal and Electron Microscopy Determinations of Cellular Fusion and Endocytosis

The confocal images were used to identified those liposomes undertaking active endocytosis with a controlled release of the encapsulated fluorescent dye in to the yeast protoplasts. Confirmation of endocytosis was confirmed by Transmission Electron Microscopy, which provided images of protoplast-liposomes fusion and both small and large liposomes bound to the cell wall as well as small liposomes found within an early endosome of a yeast cell cytoplasm.

### CONCLUSION

The action of DOTAP to facilitate coalescence between the liposomes and the yeast protoplast appears to be essential in facilitating cellular fusion while the addition of DOPE particularly provides for a greater release of the bound conjugated dextran compared to the liposomes containing phosphatidylethanolamine, however both work to various degrees. The requirement for a similar membrane structure between the formed liposome and the prepared yeast protoplast is highlighted by the need for a higher concentration of phosphatidylcholine and the inclusion of ergosterol in the liposomal matrix to facilitate the endosomal release of the fluorescent captured dye compared to the formulas with lower PC or without ergosterol as their presence allows lipid exchange and produces gaps in the 2 membrane structures.

It can be concluded then that cationic liposomes which are formed at a higher pH than the target cells pH are capable of fusing and entering a cells cytoplasm if the formulation of the liposome is similar to that of the target cells membrane.

The action of a trigger release mechanism incorporating phosphatidylethanolamine is important for the release of the components bound within the liposome however PE alone can also still provide a controlled release of the liposomal contents if the temperature and time of incubation is increased given the structural integrity of a yeast cell wall.

1. A process for producing a liposome for fusing with a target lipid layer comprising:
a) analysing a target lipid layer to determine the identity of species of lipids comprised in the target lipid layer;
b) measuring the amount of each identified species of lipid in the target lipid layer to determine the relative amount of each identified species in the target lipid layer; and
c) combining lipids of each identified species to form a liposome in which each identified species has a relative amount that is substantially the same as in the target lipid layer.

2. A process according to claim 1 wherein in step (a), the target lipid layer is analysed to determine the identity of all species of lipids comprised in the target lipid layer.

3. A process according to claim 1 wherein in step (c), the lipids of all identified species are combined to form a liposome.

4. A process according to claim 3 wherein lipids of more than one identified species that together constitute about 80% of the lipids of the target lipid layer are combined to form a liposome.

5. A process according to claim 1 wherein at least one identified species of lipid is phosphatidyl choline.

6. A process according to claim 1 wherein at least one identified species of lipid is phosphatidyl ethanolamine.

7. A process according to claim 1 wherein at least one sterol is combined with lipids of each identified species in step c) to form a liposome.
